(19) United States
(12) Patent Application Publication
(10) Pub. No.: US 2018/0235522 A1
(43) Pub. Date: Aug. 23, 2018

(54) MECHANICALLY CO-LOCATED SWEAT STIMULATION AND SENSING

(71) Applicant: University Of Cincinnati, Cincinnati, OH (US)

(72) Inventor: Jason Charles Heikenfeld, Cincinnati, OH (US)

(21) Appl. No.: 15/757,465

(22) PCT Filed: Sep. 9, 2016
(86) PCT No.: PCT/US16/50928

§ 371 Date: Mar. 5, 2018

Related U.S. Application Data
(60) Provisional application No. 62/215,816, filed on Sep. 9, 2015.

(51) Int. Cl. A61B 5/145; A61B 10/00; A61B 5/00

(52) U.S. Cl. CPC A61B 5/14521; A61B 10/0064; A61B 2562/046; A61B 5/6833; A61B 5/4266

(57) ABSTRACT

The disclosed invention provides a sweat sensor device capable of high performance stimulation and sensing at the same site on the skin, by mechanically co-locating the sensing and stimulation components when stimulation and sensing are needed, and by mechanically removing one or both of the stimulation or sensing components when stimulation and/or sensing are not needed.

### BACKGROUND OF THE INVENTION

Sweat sensing technologies have enormous potential for applications ranging from athletics, to neonatology, to pharmacological monitoring, to personal digital health, to name a few applications. Sweat contains many of the same biomarkers, chemicals, or solutes that are carried in blood and can provide significant information enabling one to diagnose ailments, health status, toxins, performance, and other physiological attributes even in advance of any physical sign. Furthermore, sweat itself, the action of sweating, and other parameters, attributes, solutes, or features on, near, or beneath the skin can be measured to further reveal physiological information.

If sweat has such significant potential as a sensing paradigm, then why has it not emerged beyond decades-old usage in infant chloride assays for Cystic Fibrosis or in illicit drug monitoring patches? In decades of sweat sensing literature, the majority of medical literature utilizes the crude, slow, and inconvenient process of sweat stimulation, collection of a sample, transport of the sample to a lab, and then analysis of the sample by a bench-top machine and a trained expert. This process is so labor intensive, complicated, and costly that in most cases, one would just as well implement a blood draw since it is the gold standard for most forms of high performance biomarker sensing. Hence, sweat sensing has not emerged into its fullest opportunity and capability for biosensing, especially for continuous or repeated biosensing or monitoring. Furthermore, attempts at using sweat to sense “holy grails” such as glucose have not yet succeeded to produce viable commercial products, reducing the publically perceived capability and opportunity space for sweat sensing.

Of all the other physiological fluids used for bio monitoring (e.g., blood, urine, saliva, tears), sweat has arguably the least predictable sampling rate in the absence of technology. However, with proper application of technology, sweat can be made to actually outperform all other non-invasive biofluids in predictable sampling. This is because you cannot easily control saliva or tear rate without consequences to the user (e.g., dry eyes, tears, dry mouth, or excessive saliva while talking). Urine is also difficult, because it is very challenging to control the amount of dilution of biomarker in urine without causing inconvenience to the user or test subject. Importantly, sampling sweat when needed, and at the right sweat rate, is further beneficial because there are biofluid secretion rates which are ideal for having the biofluid provide biomarker correlations with blood (e.g., too high of biofluid secretion will dilute a biomarker concentration as it may not have time to equilibrate by diffusion into the biofluid). An excellent summary is provided by Sonner, et al., in “The microfluidics of the eccrine sweat gland, including biomarker partitioning, transport, and biosensing implications,” Biomicrofluidics 9, 031301 (2015).

### SUMMARY OF THE INVENTION

Many of the drawbacks and limitations stated above can be resolved by creating novel and advanced interplays of mechanical elements, chemicals, materials, sensors, electronics, microfluidics, algorithms, computing, software, systems, and other features or designs, in a manner that affordably, effectively, conveniently, intelligently, or reliably brings sweat sensing and stimulating technology into intimate proximity with sweat as it is generated. With such a new invention, sweat sensing could become a compelling new paradigm as a biosensing platform.

The disclosed invention provides a sweat sensor device capable of high performance stimulation and sensing at the same site on the skin, by mechanically co-locating the stimulation and sensing components when stimulation and sensing are needed, and by mechanically removing the stimulation or sensing components when stimulation and sensing are not needed.

### BRIEF DESCRIPTION OF THE DRAWINGS

The objects and advantages of the disclosed invention will be further appreciated in light of the following detailed descriptions and drawings in which:

FIG. 1 is a top view of a portion of a wearable device for sweat biosensing according to an embodiment of the disclosed invention.

FIG. 2A is a cross-sectional view of the device taken along the line 2A-2A in FIG. 1 showing a sensing component in contact with the skin.

FIG. 2B is a cross-sectional view of the device in FIG. 2A showing a stimulating component in contact with the skin.

FIG. 3 is a cross-sectional view of the device taken along the line 3-3 in FIG. 1 showing the stimulating component in contact with the skin.

FIG. 4 is a top view of a portion of a wearable device for sweat biosensing according to an embodiment of the disclosed invention.

FIG. 5A is a cross-sectional view of the device taken along the line 5A-5A in FIG. 4 showing a sensing component and a stimulating component, neither being in contact with the skin.

FIG. 5B is a cross-sectional view of the device in FIG. 5A showing the stimulating component in contact with the skin.

FIG. 5C is a cross-sectional view of the device in FIG. 5A showing the sensing component in contact with the skin.

FIG. 6A is a cross-sectional view of a portion of a wearable device for sweat biosensing according to an embodiment of the disclosed invention showing a configuration capable of stimulating sweat.

FIG. 6B is a cross-sectional view of the device in FIG. 6A showing a configuration that is incapable of stimulating sweat.

FIG. 7A is a top view of a portion of a wearable device for sweat biosensing according to an embodiment of the disclosed invention showing a configuration that is capable of stimulating sweat.

FIG. 7B is a cross-sectional view of the device in FIG. 7A showing a configuration that is incapable of stimulating sweat.

### DEFINITIONS

As used herein, “continuous monitoring” means the capability of a device to provide at least one measurement of sweat determined by a continuous or multiple collection and sensing of that measurement or to provide a plurality of measurements of sweat over time.

As used herein, “determined” may encompass more specific meanings including but not limited to: a fact that is predetermined before use of a device; a fact that is determined during use of a device; or a fact that could be a combination of determinations made before and during use of a device.

As used herein, “sweat sampling rate” is the effective rate at which new sweat or sweat solutes originating from the sweat gland or from skin or tissue, reaches a sensor that measures a property of sweat or its solutes. Sweat sampling rate, in some cases, can be far more complex than a sweat generation rate (defined below). Times and rates are inversely proportional (rates having at least partial units of 1/seconds), therefore a short or small time required to refill a sweat volume can also be said to have a fast or high sweat sampling rate. The inverse of sweat sampling rate (1/s) could also be interpreted as a “sweat sampling interval” (s). Sweat sampling rates or intervals are not necessarily regular, discrete, periodic, discontinuous, or subject to other limitations. Sweat sampling rate can also be in whole or in part determined from solute generation, transport, advective transport of fluid, diffusion transport of solutes, or other factors that will impact the rate at which new sweat or sweat solutes reach a sensor and/or are altered by older sweat or solutes or other contamination sources. Sensor response times may also affect sampling rate.

As used herein, “sweat generation rate” is the rate at which sweat is generated by the sweat glands themselves. Sweat generation rate is typically measured by the flow rate from each gland in nL/min/gland. In some cases, the measurement is then multiplied by the number of sweat glands from which the sweat is being sampled. As used herein, “sweat stimulation” is the direct or indirect causing of sweat generation by any external stimulus such as chemical, heat, optical, electrical current, or other methods, with the external stimulus being applied for the purpose of stimulating sweat. One example of sweat stimulation is the administration of a sweat stimulant such as pilocarpine, acetylcholine, methacholine, carbachol, bethanochol, or other suitable chemical stimulant by iontophoresis, diffusion, injection, ingestion, or other suitable techniques. Some sweat stimulants last minutes, some hours or more. Generally, longer lasting sweat stimulation methods minimize re-arrangement of components during use of devices described herein. Sweat stimulation may also include sudo-motor axon reflex sweating, where the stimulation site and sweat generation site are not the same but are in close in proximity and are physiologically linked in the sweat response.

As used herein, a “sweat stimulating component” is any component or material that is capable of locally stimulating sweat to a rate greater than the natural local rate if such stimulation were not applied locally to the body. Examples of sweat stimulating components may include fluids or gels where the sweat stimulant diffuses into skin, gels where sweat stimulation is achieved by iontophoresis, needles or microneedles where sweat stimulation is achieved by transdermal injection, or any other suitable mechanisms for sweat stimulation.

As used herein, a “sweat sensing component” is any component or material that is capable of sensing sweat, a solute in sweat, a property of sweat, a property of skin due to sweat, or any other thing to be sensed that is in relation to sweat or causes of sweat. Sweat sensing components can include, for example, one or multiple sensors such as potentiometric, amperiometric, impedance, optical, mechanical, or other mechanisms known by those skilled in the art. A sweat sensing component may also include supporting materials or features for additional purposes, with non-limiting examples including local-buffering of sensor electronic signals or additional components for sweat management such as microfluidic materials.

As used herein, the term “analyte-specific sensor” or “sensor specific to an analyte” is a sensor specific to an analyte and performs specific chemical recognition of the analyte's presence or concentration (e.g., ion-selective electrodes, enzymatic sensors, electrically based aptamer sensors, etc.). For example, sensors that sense impedance or conductance of a fluid, such as biofluid, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

As used herein, “measured” can imply an exact or precise quantitative measurement and can include broader meanings such as, for example, measuring a relative amount of change of something. Measured can also imply a binary measurement, such as ‘yes’ or ‘no’ type measurements.

As used herein, “sweat sampling events” refers to the number of sweat samples per a given unit of time that are viable to be measured and that produce a measurement event of sweat. These events could be for a continuous flow of sweat and would be equivalent to sweat sampling rate. These events could be for a discontinuous flow of sweat, for example the number of times the sweat volume or sweat generation rate are adequate to make a proper sweat measurement. For example, if a person needed to measure cortisol three times per day, then the sweat flow rate would need to be adequate to provide a useful sweat cortisol measurement at least three times in the day, and other times during the day could be greater or lower than that adequate sweat flow rate.

As used herein, “mechanical co-location” refers to one or more components that can be mechanically moved or arranged in a manner that causes the components to be coupled or de-coupled to a common area of skin (i.e., one or both components are movable relative to the common area of skin), and such that the two or more components during at least one point are carried simultaneously by the device, and such that at least one component is continuously carried by the device during its use. The term “mechanical movement” includes manual movement of device components. For example, a device that places a stimulating component onto skin, removes the stimulating component from skin, and then with a separate device places a sensing component onto skin, does not meet the definition of “mechanical co-location” because neither of these components is always carried by the device, as will be further described in the disclosed invention. For a first example, the definition of “mechanical co-location” would be met by a device that carries a sweat sensing component during use of the device and integrates an iontophoretic sweat stimulating component temporarily, with the stimulating component during stimulation being coupled to at least a common portion of skin to which the sensing component is coupled. For a second example, the definition of “mechanical co-location” would be met by a device that carries a skin diffusion-based stimulating component during use of the device and integrates a sweat sensing component temporarily, with the sensing component during sensing occupying at least a portion of the stimulating component's location on skin. For a third example, the definition of “mechanical co-location” would be met by a device that carries a diffusion-based stimulating component and a sensing component during use of the device.

As used herein, within the context of mechanical co-location, the terms “co-located” or “coupled to skin” mean access to a common portion of skin and/or sweat from that common portion of skin and may or may not require direct skin contact (e.g., a stimulating component could directly contact the skin or could have a sweat wicking component between the sweat stimulating component and the skin). Further, a component being “in contact with skin” does not necessarily mean in direct contact with skin (i.e., there may be intervening layers). It will be made further clear based on the above examples, that the component that requires most time of placement on skin is most likely the component carried by the device during its operation, although the disclosed invention is not so limited.

### DETAILED DESCRIPTION OF THE INVENTION

The disclosed invention provides a sweat sensor device capable of stimulation and sensing at the same site, by mechanically co-locating the sweat stimulating and sensing functions of the device. The disclosed invention applies at least to any type of sweat sensor device that stimulates and measures sweat, its solutes, solutes that transfer into sweat from skin, a property of or things on the surface of skin, or properties or things beneath the skin. The disclosed invention applies to sweat sensing devices which can take on forms including patches, bands, straps, portions of clothing, wearables, or any suitable mechanism that reliably brings sweat stimulating, sweat collecting, and/or sweat sensing technology into intimate proximity with sweat as it is generated. Some embodiments of the disclosed invention utilize adhesives to hold the device near the skin, but devices could also be held by other mechanisms that hold the device secure against the skin, such as a strap or embedding in a helmet. Certain embodiments of the disclosed invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a sweat sensor; an impedance sensor; a sweat volume sensor; a sweat generation rate sensor; and a solute generation rate sensor. Certain embodiments of the disclosed invention show sub-components of what would be sweat sensing devices with more sub-components needed for use of the device in various applications, which are obvious but not necessarily critical to inventive step (such as a battery, or a counter electrode for iontophoresis), and for purpose of brevity and focus on inventive aspects are not explicitly shown in the diagrams or described in the embodiments of the disclosed invention. For example, sweat stimulating components may require an electrode for iontophoresis delivery, a gel containing the sweat stimulant, a connection to an electrical current source, and possibly other components, but in the disclosed such components may be diagramed and referred as simply a “stimulating component”.

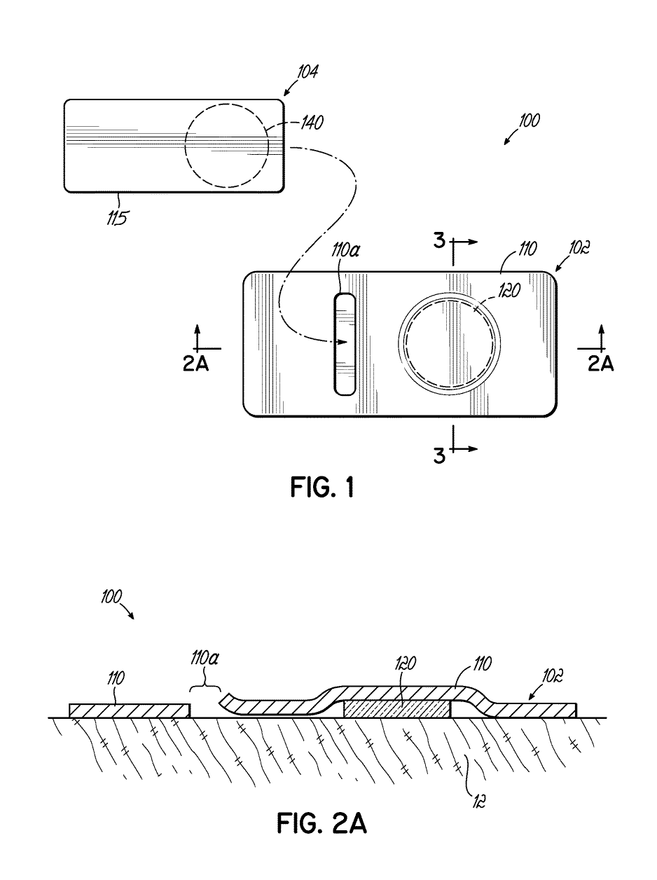

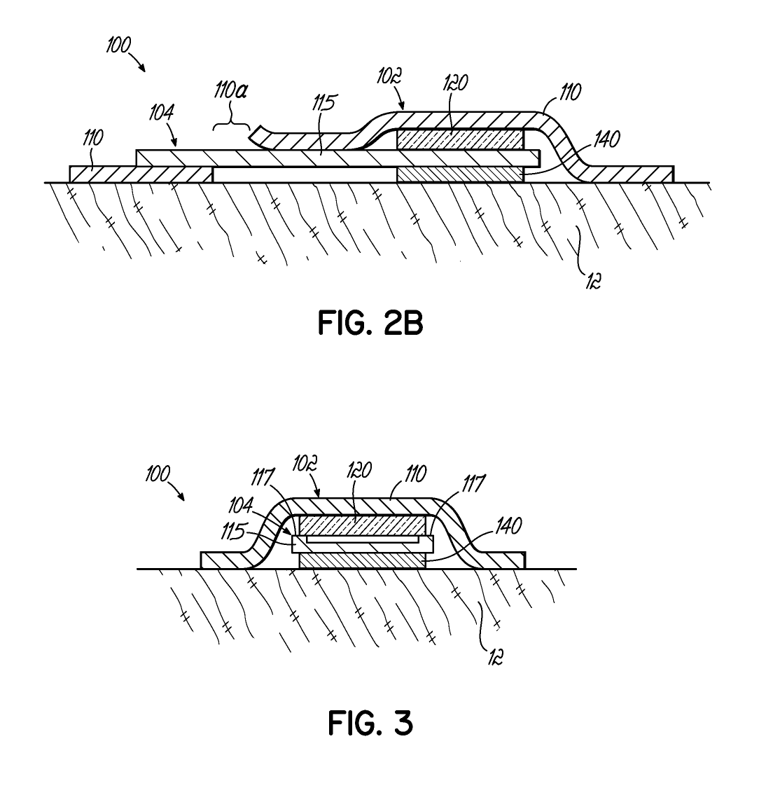

With reference to FIGS. 1 and 2A, a sweat sensing device 100 includes a sensing portion 102 and a stimulating portion 104 that are separable from each other. The sensing portion 102 includes a first substrate 110 having an aperture 110a and a sensing component 120 on the first substrate 110. In an embodiment, the first substrate 110 may be a flexible plastic film (e.g., PET) or a textile carrying the sensing component 120, which may be, for example, an electrical impedance antibody sensor for cortisol. Further, the first substrate 110 may include an adhesive suitable for adhering the device 100 to the skin 12. The stimulating portion 104 includes a second substrate 115 coupled to a stimulating component 140. In an embodiment, the second substrate 115 may be a semi-rigid plastic film, and the stimulating component may include, for example, an iontophoresis electrode carrying a semi-rigid aragose gel containing a chemical sweat stimulant. As shown in FIG. 2B, at least a portion of the stimulating portion 104 is insertable into the aperture 110a. As shown in FIG. 2A, when the device 100 is positioned on the skin 12, the sensing component 120 is near or intimate with the skin 12.

With reference to FIGS. 2B and 3, a portion of the stimulating portion 104 has been inserted through the aperture 110a. The stimulating component 140 has been moved to a co-located position on the skin 12 where sensing component 120 previously was. In other words, the stimulating component 140 is in contact with or proximate to a portion of the skin 12 that was previously in contact with or proximate to the sensing component 120. Stimulating component 140 can then stimulate sweat by, for example, iontophoresis of a sweat stimulant. Once sufficient sweat has been stimulated, the stimulating portion 104 of the device 100 may be removed to return the device 100 to the configuration shown in FIG. 2A where the sensing component 120 is able to sense the sweat that has been stimulated. This process can be repeated as needed, regularly, based on need for sweat and/or a measurement, or as determined by any method or schedule. For example, if the stimulating component 140 iontophoretically delivers carbachol, which can induce high sweat rates for numerous hours (e.g., 6 hours), the stimulation could be applied for 2 minutes (using the configuration shown in FIG. 2B) while the sensing component 120 measures sweat for approximately 5 hours and 58 minutes (using the configuration shown in FIG. 2A). The movement of the stimulating portion 104 into the aperture 110a of the sensing portion 102 can be achieved by the user (e.g., using fingers or a specially designed applicator) or by mechanical motors and tracks or other mechanical techniques (not shown) that could be integrated with the device 100.

With further reference to FIGS. 1-3, the stimulating component 140 and the sensing component 120 may be alternately configured so that these components are interchanged in their location in the device 100. Sensing component 120 would accordingly be mounted on the second substrate 115 and stimulating component 140 would be mounted on the first substrate 110. In such configuration, the stimulating component 140 may be left stationary and the sensing component 120 moved similar to the above teachings for FIGS. 1-2B. Furthermore, although not explicitly shown, both the stimulating component 140 and sensing component 120 may also be independently movable using principles of the disclosed invention, as long as they satisfy the general definition of mechanical co-location as described herein.

With further reference to FIG. 2A, the first substrate 110 is stretchy or flexible or stretches the skin 12 to hold the sensing component 120 against the skin 12 and, with reference to FIG. 2B, when the stimulating portion 104 is inserted through the aperture 110a, to hold the stimulating component 140 against the skin 12. Although not shown, springs, sponges, or other suitable methods may be used to provide pressure to secure one or more components against the skin 12. In an aspect of the disclosed invention, materials, features, or methods may be used to protect the sensing component 120 and/or the stimulating component 140 from significant damage during movement between the sensing portion 102 and the stimulating portion 104. For example, as shown in FIG. 3, the second substrate 115 includes raised portions 117, which act as a sensor-shielding component to reduce scraping or abrasion of the second substrate 115 with at least a portion of the sensing surface of sensing component 120 during mechanical movement of one or both of the components. Other sensor-shielding components may be used, such as placing textiles or microfluidics between the sensing component 120 and the skin 12 or the second substrate 115.

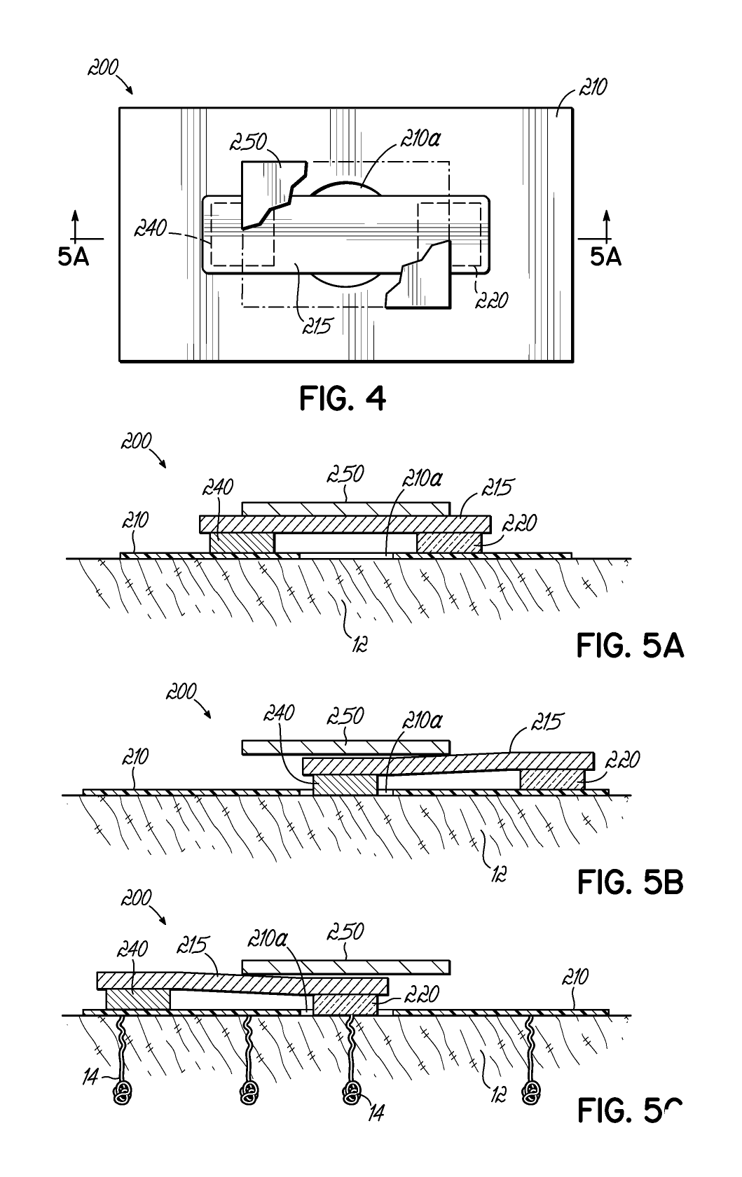

With reference to FIGS. 4 and 5A, where similar numerals refer to similar features shown and described in connection with FIG. 1, in an embodiment of the disclosed invention, a device 200 includes a sensing portion, shown as the sensing component 220, and a stimulating portion, shown as the stimulating component 240, that are each carried by the device 200 during use. The first substrate 210 includes an aperture 210a, which allows the sensing component 220 or the stimulating component 240 to contact a portion of the skin 12. In this embodiment, the second substrate 215 optionally carries both the sensing component 220 and the stimulating component 240. A third substrate 250 provides pressure to hold the sensing component 220 or the stimulating component 240 against the skin 12 during use of the device 200. The pressure provided by the third substrate 250 is not great enough to prevent movement of the second substrate 215.

With reference to FIGS. 5A-5C, the second substrate 215 may be moved from an inactive configuration (FIG. 5A) to a stimulating configuration (FIG. 5B) and to a sensing configuration (FIG. 5C). As shown in FIG. 5A, the device 200 has an inactive configuration where neither the stimulating component 240 nor the sensing component 220 is in contact with skin 12. The second substrate 215 may be moved to a stimulating configuration, as shown in FIG. 5B, where the stimulating component 240 is in contact with the skin 12. Additionally, as shown in FIG. 5C, the second substrate 215 may be moved to a sensing configuration where the sensing component 220 is in contact with the skin 12 and is able to measure sweat generated from the sweat glands 14. Thus, in use, the configuration of the device 200 may be adjusted between the inactive, stimulating, and sensing configurations as needed. For example, in an embodiment where the stimulating component 240 includes carbachol contained in a glycol-filled sponge, the stimulating component 240 may be in contact with the skin 12 for two hours during which the carbachol diffuses through the skin 12 to stimulate sweat. Sensing component 220, which could be a sensor for lead exposure, may be applied to the skin 12 once every two hours. As a result, multiple readings of lead in sweat can be implemented. In an alternate embodiment, the device 200 could be operated similar to the exemplary operation of the device 100 described above (i.e., using iontophoresis, and the sensing component 220 spending more time on the skin 12 than the stimulating component 240). In an embodiment, the mechanical movement of the second substrate 215 could be automated (e.g., using motors and controls) or manual (e.g., caused by the user applying horizontal force to the second substrate 215).

In an aspect of the disclosed invention, various components can be independently operating or interconnected. For example, a sensing component could include a battery, be equipped for Bluetooth wireless communication, interconnects between sensors and electronics, etc. In another example, a stimulating component may be an iontophoresis unit that includes electronics to self-terminate the application of iontophoresis after a dose is provided. As a further example, a stimulating component could be integrated with other electronics on the device through a single electrical lead needed to drive the iontophoresis process. As a further example, a sensing component could have one or more wired and flexible connections to electronics on the device, which flexes as mechanical movement occurs. In another example, sliding or temporary electrical contact pads between sensors and electronics may be used so long as they are kept dry or insulated from sweat using a suitable method such as the use of grease or a wicking component to keep sweat away from the exposed electrical contacts. For example, electrical contact to the sensor component 220 or stimulating component 240 could be formed automatically as either component is moved into contact with the skin 12.

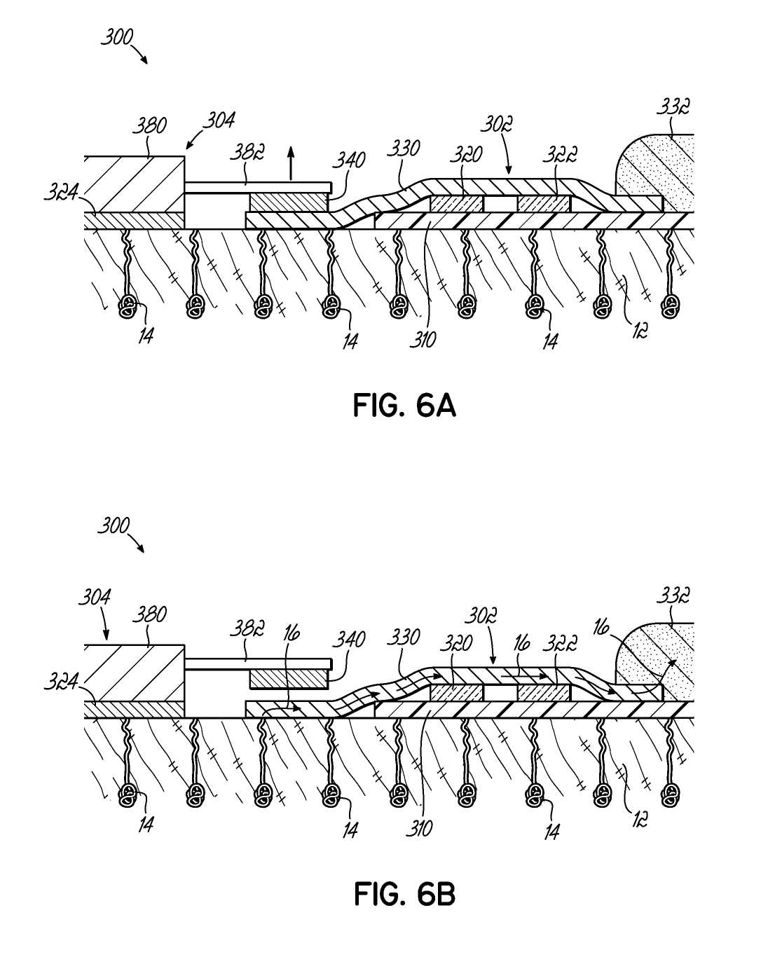

With reference to FIGS. 6A and 6B, where similar numerals refer to similar features shown and described in connection with FIG. 4, in an embodiment of the disclosed invention, a device 300 includes a sensing portion 302 and a stimulating portion 304. The sensing portion 302 includes a polymer substrate 310 that carries sensors 320, 322. In an embodiment, the sensor 320 may be an amperometric sensor for sensing urea, and the sensor 322 may be an aptamer-based sensor for vasopressin or a thermal-based flow sensor for measuring sweat flow rate and therefore determining sweat generation rate. The stimulating portion 304 further includes an electrode 324 for measuring skin impedance, which could alternatively be any type of sensor for determining the presence of naturally generated sweat. For example, a sensor 324 could be an amperometric lactate sensor because lactate increases in sweat with increasing sweat generation rate. As shown by the arrows 16, a wicking component 330 transports sweat from the skin 12, past the sensors 320, 322 where the sweat is measured, and eventually into the wicking pump 332, which collects excess and old sweat. In an example, the wicking component 330 may be paper, and the wicking pump 332 may be a water absorbing polymer such as, for example, a hydrogel. Sweat flow rate sensor 322 and electrode 324 can be used in tandem to determine the combined amount of natural sweating and the amount of stimulated sweating, thereby informing the device 300 how often and/or how much sweat stimulation is needed.

With further reference to FIGS. 6A and 6B, the stimulating portion 304 includes a stimulating component 340 that is coupled to an arm 382, which is mechanically movable, for example, by a magnetic solenoid actuator 380. Thus, the actuator 380 moves the arm 382 to initiate any coupling (e.g., fluidic, thermal, chemical, or other suitable coupling for stimulation) between the stimulating component 340 and the skin 12. An active configuration of the stimulating portion 304 is shown in FIG. 6A where the stimulating component 340 is in contact with the wicking component 330. Because the wicking component 330 is porous to the sweat stimulant and sweat, the wicking component 330 fluidically couples the stimulating component 340 to the skin 12, and the stimulating component 340 is able to stimulate sweat. Therefore, even during sweat stimulation, there does not need to be direct skin contact between the stimulating component 340 and the skin 12. In that regard, one or more coupling components (i.e., the wicking component 330 acts as a fluidically coupling component) may be positioned between the stimulating component 340 and the skin 12. In FIG. 6B, an inactive configuration of the stimulating portion 304 is shown where the stimulating component 340 is mechanically removed from contact with the wicking component 330. The actuator 380 moves the arm 382 to terminate any coupling between the stimulating component 340 and the skin 12. It should be recognized that the actuator 380 and the arm 382 may be replaced with other components suitable to bring the stimulating component 340 in and out of contact with the wicking component 330. For example, suitable mechanical actuation components that may be used in embodiments of the disclosed invention include various types of motors and all known techniques used for artificial muscles (e.g., electro-active polymers, piezo-electric, thermal actuators, etc.).

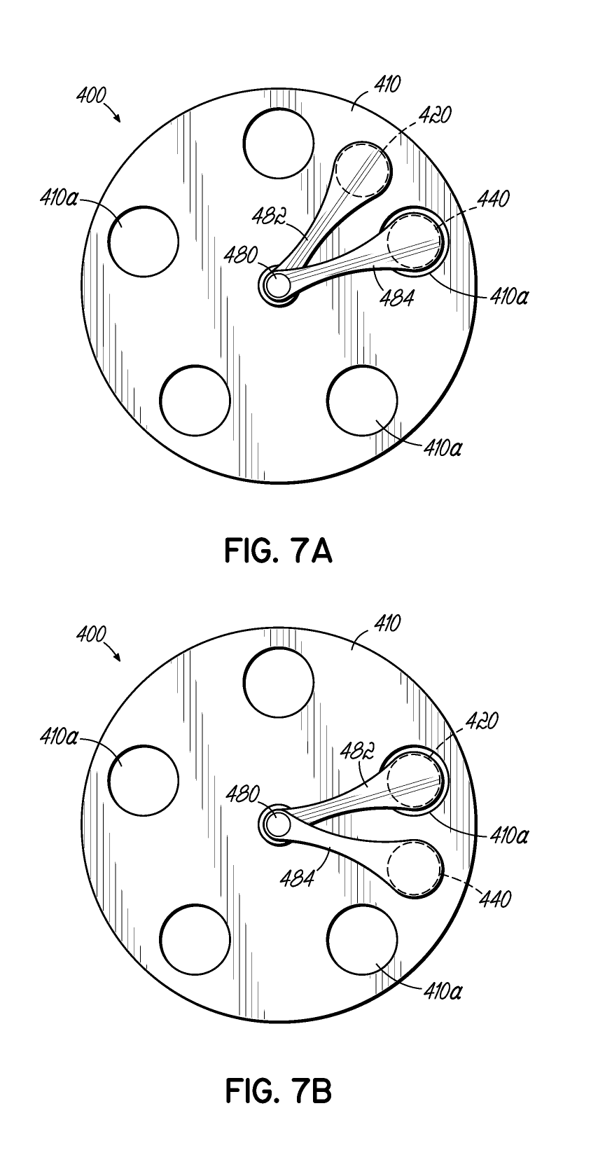

With reference to FIGS. 7A and 7B, where similar numerals refer to similar features shown and described in connection with FIG. 4, in an embodiment of the disclosed invention, a device 400 includes a polymer 410 having apertures 410a that provide access to the skin (not shown) when device 400 is placed on the skin. The device 400 includes a rotary movement system 480, a first arm 482, and a second arm 484. At least one sensing component 420, which is specific to an analyte in sweat, is coupled to the first arm 482, and at least one stimulating component 440 is coupled to the second arm 484. A stimulating configuration is shown in FIG. 7A where the stimulating component 440 is in contact with the skin to stimulate sweat. A sensing configuration is shown in FIG. 7B where the stimulating component 440 is mechanically moved to bring the sensing component 420 into contact with the site on the skin where sweat was stimulated, which therefore allows sensing of at least one analyte in sweat. The movement of the sensing component 420 and the stimulating component 440 could be achieved by activating the rotary movement system 480 to rotate the first and second arms 482, 484, which could be made of plastic, metal, or another suitable material. In an embodiment, the rotary movement system 480 may include radial gear coupled to a linear gear and a linear gear actuator.

With reference to FIGS. 7A and 7B, in the illustrated embodiment, at any given time, only one of the sensing component 420 or the stimulating component 440 can contact the skin (i.e., both cannot be in contact with the skin simultaneously). However, the apertures 410a may be arranged such that at least one sensing component 420 and at least one stimulating component 440 could be in contact with the skin at the same time. Further, in an embodiment, the apertures 410a may be arranged to allow an inactive configuration where neither the sensing component 420 nor the stimulating component 440 is in contact with the skin.

Several uses of the device 400 are now described. For example, the stimulating component 440 may be used to stimulate sweat, and the device 400 may be adjusted to be in the inactive configuration for 30 minutes before moving to a sensing configuration. This would allow sweat to not be sensed until 30 minutes after stimulation, if, for example, the stimulation caused skin swelling or irritation for 20-30 minutes, and the sensing component 220 is configured to provide a one-time measurement of pro-inflammatory analytes. In another embodiment, the stimulating component 440 could move independently of the sensing component 420. Thus, the stimulating component 440 could stimulate one or more sites on the skin 12 with one or more sweat generation rates. For example, the stimulating component 440 may stimulate sweat at a generation rate of 0.5 nL/min/gland on a first skin site and stimulate sweat at a generation rate of 5 nL/min/gland on a second skin site, thus allowing the sensing component 420 to sense sweat at different sweat rates to determine, for example, the amount of dilution of vasopressin by ultrafiltration in sweat and therefore improve quantitative analysis of vasopressin. It should be recognized that aspects of the disclosed invention can be combined or altered in numerous ways. For example, the sensing component 420 of the device 400 could be replaced by the wicking component 330 of the device 300, which transports sweat to one or more sensors 320, 322.

The following examples are provided to help illustrate the disclosed invention, and are not comprehensive or limiting in any manner.

### EXAMPLE 1

With reference to FIGS. 4-5C, a person wearing the device 200 desires to measure cortisol levels in sweat only during awakening and during stressful events. As the person wakes up, the person may manually slide the second substrate 215, so that the stimulating component 240 comes into contact with the skin 12. The device 200 may be configured to sound an auditory alert to let the user know to move the sensing component 220 into place to measure sweat cortisol levels as the person continues to awaken. Throughout the day, when the person feels stressed, the person may manually move the second substrate 215 into a stimulating configuration and, subsequently, into a sensing configuration.

### EXAMPLE 2

A group of workers wishes to monitor themselves for lead (Pb) exposure. The workers each wear a device that alternately stimulates sweat through transdermal diffusion of a sweat stimulant, and measures for Pb in sweat every 2 hours. This occurs automatically and the devices include a motor and moveable track that positions the sensing and stimulating portions as needed.

What is claimed is:

1. A device for sensing sweat on skin, comprising:
at least one sensor that is specific to an analyte in the sweat;
at least one sweat stimulating component for stimulating sweat on an area of the skin; and
wherein said sensor and said sweat stimulating component are mechanically co-located on at least a portion of said sweat stimulation area for at least a portion of time during device use.

2. The device of claim 1, wherein the device includes an active configuration where at least one of the at least one sensor or the at least one sweat stimulating component is positioned to be coupled with skin.

3. The device of claim 1, wherein the device includes an inactive configuration where neither the at least one sensor nor the at least one sweat stimulating component is positioned to be coupled with skin.

4. The device of claim 1, further comprising at least one sensor-shielding component to reduce abrasion of the sensor during mechanical movement of the sensor or the sweat stimulating component.

5. The device of claim 1, further comprising at least one of a sensor for measuring stimulated sweat generation rate, a sensor for measuring stimulated sweat flow rate, or a sensor for measuring stimulated sweat sampling interval.

6. The device of claim 5, further comprising at least one of a sensor for measuring natural sweat generation rate, a sensor for measuring natural sweat flow rate, or a sensor for measuring natural sweat sampling interval.

7. The device of claim 1, further comprising at least one coupling component between said at least one stimulating component and said sweat stimulation area.

8. The device of claim 1, further comprising at least one sweat wicking component between said at least one stimulating component and said sweat stimulation area.

9. The device of claim 1, wherein said at least one sensor provides a plurality of measurements specific to said analyte in the sweat.

10. The device of claim 1, further comprising a sweat impermeable substrate having a plurality of apertures that provide access to a plurality of areas of the skin.

11. A device to measure one or more components of sweat, comprising:
a sweat stimulating component for stimulating sweat on an area of the skin; and
a sensor,
wherein said sweat stimulating component and said sensor are moveable relative to the sweat stimulation area.

12. The device of claim 11, wherein said sweat stimulating component is carried on a first substrate and said sensor is carried on a second substrate, wherein the first substrate may move relative to the second substrate.

13. The device of claim 12, wherein the first substrate and the second substrate are coupled to a third substrate by a rotary movement system.

14. The device of claim 11, wherein said sensor and said sweat stimulating component are carried on a first substrate, and said first substrate is carried on a second substrate, where the first substrate may move relative to the second substrate.

15. The device of claim 14, wherein the first substrate is coupled to the second substrate by a rotary movement system.
